(19) United States
(12) Patent Application Publication
(10) Pub. No.: US 2014/0060543 A1
(43) Pub. Date: Mar. 6, 2014

(54) Apparatus for Resuscitation near MRI Chambers

(71) Applicant: MERCURY ENTERPRISES, INC., Clearwater, FL (US)

(72) Inventors: George E. Howe, JR., Largo, FL (US); Brian Maxfield, Clearwater, FL (US); Jeffrey B. Ratner, Pinellas Park, FL (US)

(73) Assignee: MERCURY ENTERPRISES, INC., Clearwater, FL (US)

(21) Appl. No.: 14/025,337

(22) Filed: Sep. 12, 2013

Related U.S. Application Data
(63) Continuation-in-part of application No. 12/838,555, filed on Jul. 19, 2010, now abandoned.

(51) Int. Cl. A61M 16/20; A61M 16/00

(52) U.S. Cl. CPC A61M 16/20; A61M 16/0057
USPC 128/205.24

(57) ABSTRACT

An application for a disposable support/resuscitation system is disclosed includes a pressurized gas inlet and a pressure relief device interfaced to the pressurized gas inlet. The pressure relief device has a first pressure relief valve that opens at a setable gas pressure and, optionally, has a second pressure relief valve that opens at a pre-determined maximum gas pressure. A manometer is connected to the pressure relief valve. A manually operated valve is connected/interfaced to the manometer, and a patient interface port is connected/interfaced with the manually operated valve. The manually operated valve selectively controls administration of the pressurized gas to the patient and both the manometer and the manually operated valve are in close proximity to the patient. The manometer, pressure relief device, and manually operated valve are made from non-ferromagnetic materials for proper operation in the vicinity of a Magnetic Resonance Imaging System.

### CROSS-REFERENCE TO RELATED APPLICATION

This application is a continuation-in-part of U.S. patent application Ser. No. 12/838,555, filed Jul. 19, 2010, the disclosure of which is hereby incorporated by reference.

### FIELD

This invention relates to the field of resuscitation and more particularly to a disposable system, method and apparatus for resuscitating a person, perhaps an infant, in the vicinity of a magnetic resonance imaging (MRI) system.

### BACKGROUND

In situations when a patient has a cardiac arrest or ceases to breath, emergency life support and/or resuscitation requires a way to supplement and hopefully revive the patient's breathing function. When equipment is unavailable, often the life support and/or resuscitation is performed by administration of Cardio-Pulmonary Resuscitation techniques, or CPR.

In situations when equipment is available, such as in a hospital, life support and/or resuscitation are often accomplished by the use of a manually operated resuscitation device. These manually operated devices are fed with oxygen (or other breathable gases such as air) under pressure that is administered to the patient through a mask or tracheal tube, Administration is under the control of an administrator such as a doctor or a nurse. The administrator controls the flow and abatement of the oxygen to the patient, filling the patients lungs, then stopping the flow of oxygen, at which time the patient exhales.

Manometers for measuring gas pressure in a patient ventilation system are well known. U.S. Pat. No. 5,557,049 to Jeffrey B. Ratner describes a Manometer for insertion into a patient ventilation system and is herein included by reference.

There are several problems that prior life support/resuscitation systems and devices need overcome. The first problem is to limit the gas pressure so as not to over inflate the patient's lungs and possibly causing a rupture. The second problem is to provide feedback to the administrator to inform the administrator of the pressure within the breathing system and when the patient starts breathing on their own. Another issue relates to sterility of the life support/resuscitation systems and devices when used on the next patient.

Another problem that needs to be overcome is using the device in the vicinity of magnetic resonance imaging (MRI) systems. Due to the strong magnetic fields created within and around these magnetic resonance imaging (MRI) systems, existing resuscitation systems are inadequate because several components such as non-ferromagnetic resilient members and shafts are typically made out of materials that are attracted by the magnetic forces generated by magnetic resonance imaging (MRI) systems, thereby causing erroneous readings on, for example, manometers and, in extreme cases, movement of the resuscitation devices under the pull of the magnetic resonance imaging (MRI) system.

What are needed are support/resuscitation systems and devices that will provide control and status to the administrator at the patient locale and permit disposability.

### SUMMARY

In one embodiment, a disposable support/resuscitation system is disclosed including a pressurized gas inlet and a pressure relief device interfaced to the pressurized gas inlet. The pressure relief device has a first pressure relief valve that opens at a setable gas pressure and has a second pressure relief valve that opens at a pre-determined maximum gas pressure. A manometer is interfaced to the pressure relief valve, a manually operated valve is interfaced to the manometer, and a patient interface port is interfaced with the manually operated valve. The manually operated valve selectively controls administration of the pressurized gas to the patient and both the manometer and the manually operated valve are in close proximity to the patient. Close proximity is a term used to mean that both the manometer and the manually operated valve are close enough to the patient that a caregiver need not look away or turn away from the patient to operate the manually operated valve or to read the current gas pressure from the manometer. All sub-components of the disposable support/resuscitation system are fabricated from non-ferromagnetic materials.

In another embodiment, a disposable support/resuscitation system is disclosed including a pressurized gas inlet and a pressure relief device that is interfaced to the pressurized gas inlet. The pressure relief device has a valve for adjustably regulating gas pressure and a valve for regulating the gas pressure below a pre-determined maximum gas pressure. There is a device for displaying the gas pressure and a device for modulating the gas pressure, both interfaced to the pressure relief valve. A patient interface port is connected to the device for displaying the gas pressure and to the device for modulating the gas pressure and provides modulated gas pressure to a patient. The device for modulating the gas pressure selectively controls administration of the gas pressure to the patient and both the device for displaying and the device for modulating the gas pressure are in close proximity to the patient. Close proximity is a term used to mean that both the device for modulating the gas pressure and the device for displaying the gas pressure are close enough to the patient that a caregiver need not look away or turn away from the patient to modulating the gas pressure or to read the current gas pressure from the device for displaying the gas pressure. All sub-components of the disposable support/resuscitation system are fabricated from non-ferromagnetic materials.

In another embodiment, a disposable support/resuscitation system is disclosed including a pressure relief device that has an (e.g. industry standard) gas inlet and a gas output connector. The pressure relief device has a first pressure relief valve that opens at a setable gas pressure and a second pressure relief valve that opens at a pre-determined maximum gas pressure. The disposable support/resuscitation system includes a manometer and a gas delivery tube that fluidly connects the gas output connector to the manometer. A manually operated valve is also fluidly connected to the manometer and a patient interface port is connected to the manually operated valve. The manually operated valve selectively controls administration of pressurized gas from the gas inlet to the patient. The manometer and the manually operated valve are in close proximity to the patient to provide more accurate pressure readings, reduce administrator fatigue and reduce the need to look away from the patient. Close proximity is a term used to mean that both the manometer and the manually operated valve are close enough to the patient that a caregiver need not look away or turn away from the patient to operate the manually operated valve or to read the current gas pressure from the manometer. All sub-components of the disposable support/resuscitation system are fabricated from non-ferromagnetic materials.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a schematic view of a resuscitation system of the prior art.

FIG. 2 illustrates a perspective view of a disposable resuscitation system.

FIG. 3 illustrates a view of the disposable resuscitation system in use in conjunction with an infant face mask.

FIG. 4 illustrates a view of the disposable resuscitation system in use in conjunction with an infant tracheal tube.

FIG. 5 illustrates a sectional view of a pressure relief device of the disposable resuscitation system.

FIG. 6 illustrates an exploded view of a pressure relief device of the disposable resuscitation system.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures. Throughout this document, the term “close proximity to the patient” means that the devices listed are close enough to the patient as to be monitored and operated without having to move away from the patient and/or without having to look away from the patient. This is important, for instance, when a patient is being resuscitated and it is important to constantly monitor the patient's color, breathing and the pressure in their lungs.

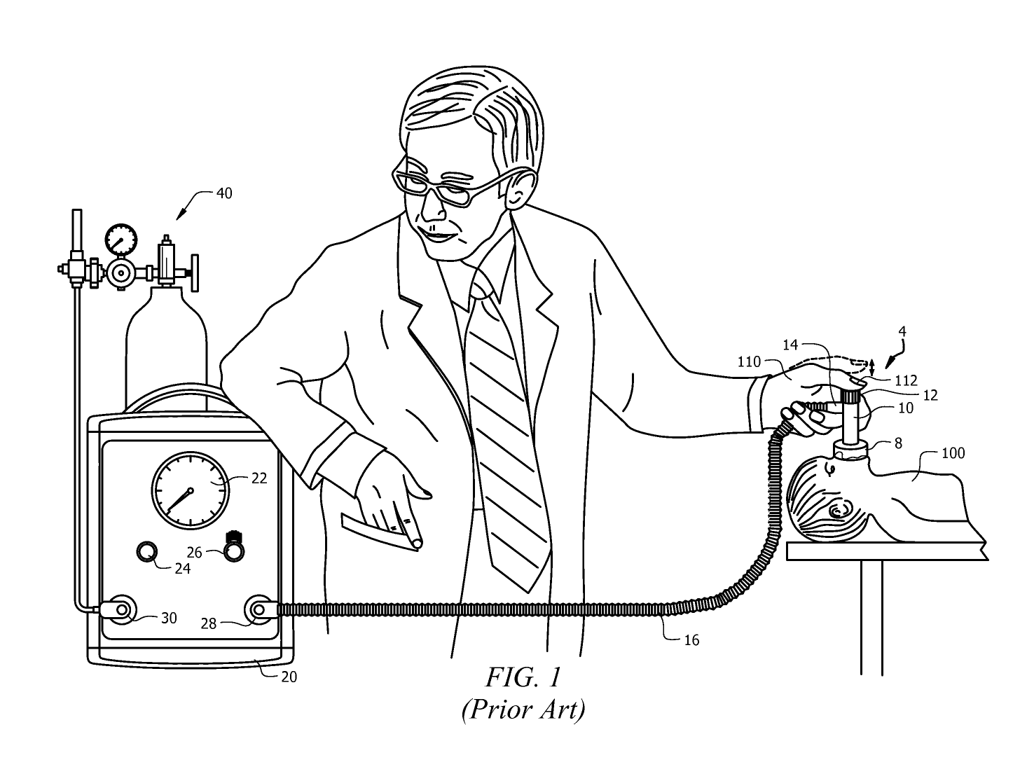

Referring to FIG. 1, a schematic view of a resuscitation system of the prior art is shown. Resuscitation systems have a source of pressurized gas (e.g. pressurized air, oxygen, etc) such as an oxygen tank system 40. Such sources of pressurized gas are well known and deliver sufficient gas pressure as to inflate a lung of a patient. The pressurized gas is fluidly coupled to an inlet 30 of a gas pressure control device 20. Within the oxygen flow control device 20, fluid pressure is monitored by a manometer 22 and a pressure is controlled by a maximum pressure valve 24 and a pressure adjustment valve 26. The resulting controlled pressure gas exits from a gas outlet 28 through a gas delivery tube 16 that is often significant in length to reach the patient 100. The gas delivery tube 16 is connected to a T-piece device 4 at an inlet port 14 and delivered to the patient 100 through a patient delivery port 10 that is connected to, for example, a face mask 8 covering the patient's mouth and nose. An adjustable finger valve 12 is operated by a finger 112 of the administration person 110 (e.g. doctor or nurse). The administrator 110 presses their finger 112 against the opening of the finger valve 12 to inflate the patient's 100 lungs and removes their finger 112 from the finger valve 12 to let the patient 100 exhale. In order to see the pressure reading on the manometer 22, the administrator 110 looks away from the patient 100. This distracts from carefully monitoring the patient 100 to observe lung activity, patient skin tone, obstructions to the air flow, etc.

Additionally, only the gas delivery tube 16 (e.g. single-use patient supply lines), the T-piece device 4 and the face mask 8 (or tracheal tube—not shown) are disposable. Biological or chemical agents that make their way back into the gas pressure control device 20 are subject to be delivered, inadvertently, to the next patient since the gas pressure control device 20 is not disposable and is not easily sterilized. User manuals for some gas pressure control devices 20 include cleaning and service steps that only address cleaning and drying external surfaces. Should gas pressure from the source of pressurized gas drop suddenly (e.g. from a hospital supply system), back pressure from the patient's 100 lungs may push chemical or biological agents back into the gas pressure control device 20 and such may get inadvertently delivered to the next patient. The gas pressure control device 20 is not disposable and there is no apparent way to sterilize gas pressure control devices 20 between patients.

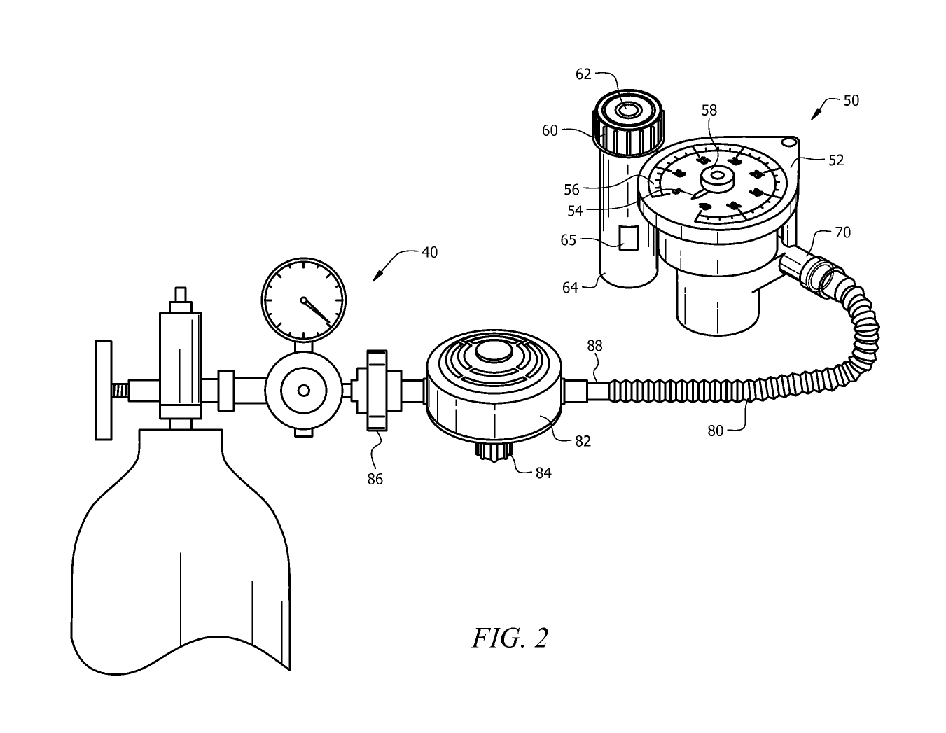

Referring to FIG. 2, a perspective view of a disposable resuscitation system 50 for use in the vicinity of an MRI system is shown. Resuscitation systems have a source of pressurized gas 40 (e.g. pressurized air, oxygen, etc) such as an oxygen tank system 40. Such sources of pressurized gas are well known and deliver sufficient gas pressure as to inflate a lung of a patient.

The pressurized gas is fluidly coupled to an inlet of a pressure relief device 82 through a gas input coupling 86 as known in the industry. For use in the vicinity of an magnetic resonance imaging (MRI) system, it is anticipated that the source of pressurized gas 40 is located away from the magnetic resonance imaging (MRI) system, perhaps in a different room, and is coupled to the pressure relief device 82 through tubing, preferably non-ferromagnetic tubing.

The pressure relief device 82 has one adjustable pressure relief valve that is controlled by an adjustment knob 84 and a second, fixed pressure relief valve that releases pressure at a pre-determined maximum pressure, thereby not permitting an output gas pressure to exceed the pre-determined pressure.

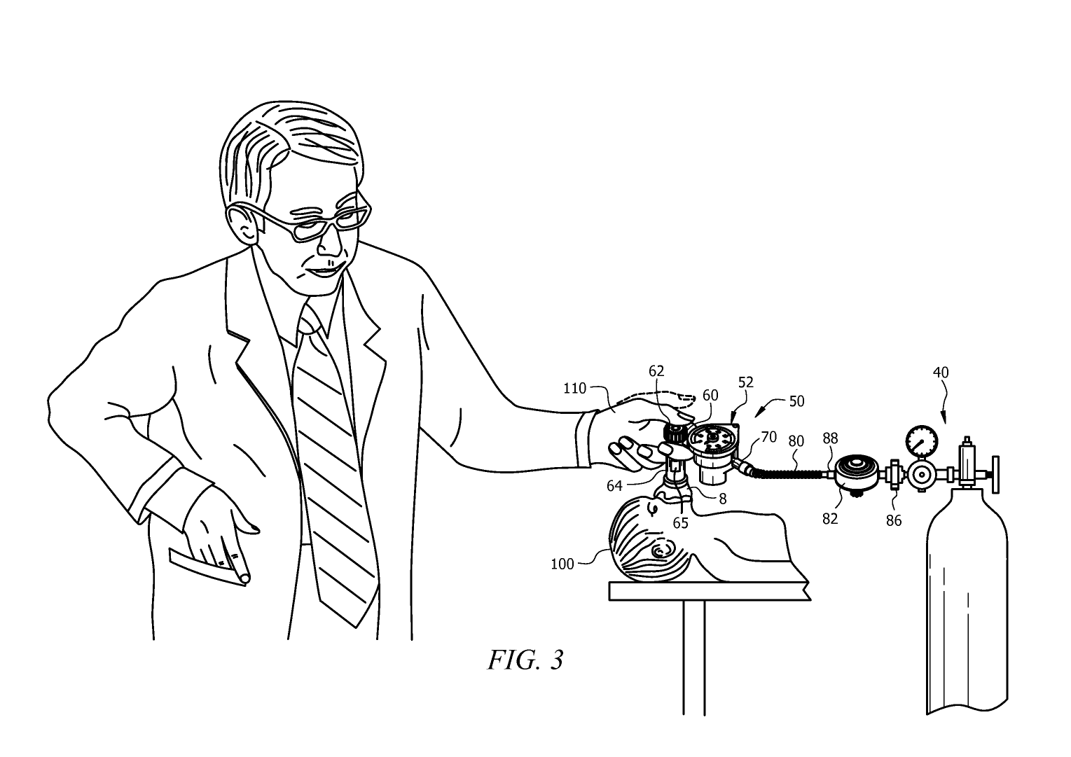

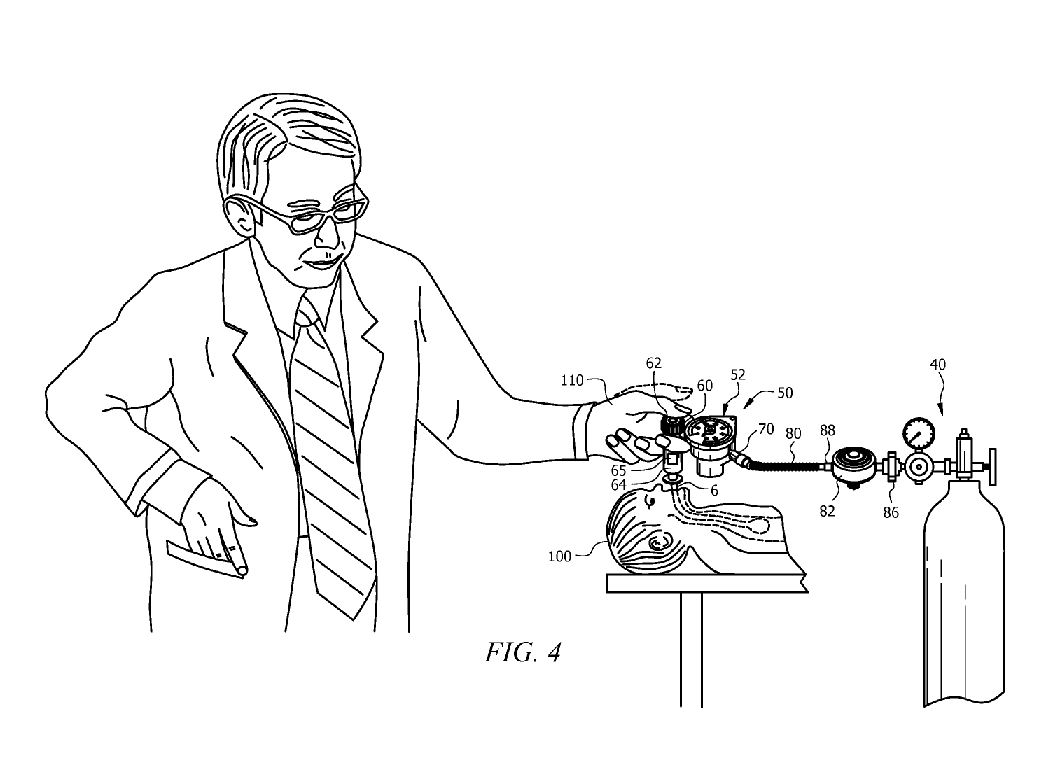

The pressure relief device 82 is in fluid communication with a manometer 52 (pressure meter) and T-piece valve assembly 60/62/64. In some embodiments, a colorimetric carbon dioxide detector 65 is in fluid communication with the patient interface port to detect proper intubation. A section of gas delivery tube 80 connects an output connector 88 on the pressure relief device 82 to an inlet port 70 of the T-piece valve assembly. The pressurized gas is then in fluid communication with the manometer 52, the finger valve 60/62 and the patient port 64. The patient port 64 is then interfaced to the patient 100 through, for example, a face mask 8 (see FIG. 3) or a tracheal tube 6 (see FIG. 4). The manometer 52 has an indicator 54 that moves around a hub 58 responsive to pressure values of the pressurized gas, pointing to gradients 56 indicative of the pressure at the patient 100. The finger valve 60/62 is operated by, for example, a finger 112 of the administrator 110. When finger 112 is pressed against the valve opening 62, pressure increases and the patient's 100 lungs inflate and the pressure level is shown on the manometer 52. When the finger 112 is released from the valve opening 62, the pressure abates and the patient 100 exhales through the valve opening 62. In some embodiments, the valve 60/62 is adjustable by turning the knob 60 to increase or decrease back pressure as the patient exhales. Such valves are known in the industry and any such valve that is operated by the administrator 110 is anticipated.

Although a finger operated valve 60/62 is shown and preferred, any known valve is anticipated for modulating the gas pressure to the patient 100 including mechanical valves, electrically controlled valves, etc.

U.S. Pat. No. 5,557,049 to Jeffrey B. Ratner describes a manometer for insertion into a patient ventilation system and is herein included by reference, though the disclosed manometer in 5,557,049 has metal, ferromagnetic resilient members that are not compatible with MRI systems. In this, the strong magnetic field of the MRI system will act upon the ferromagnetic resilient members within the manometer, generating false readings or, even worse, dislocate the manometer, potentially causing bodily harm. To overcome this problem, the manometer 50 is made without the inclusion of any ferromagnetic materials, in such the non-ferromagnetic resilient member (not visible), shaft (not visible), dial 54, and all other components are made of a suitable, non-ferromagnetic material such as plastic.

In some manometer/T-piece valve systems, a colorimetric carbon dioxide indicator 65 is disposed in the exhalation path. The colorimetric carbon dioxide indicator changes color under the presence of carbon dioxide and, since living beings exhale carbon dioxide, the color change is useful in determining that the patient is exhaling, indicating that a tracheal tube is properly inserted into the airway as opposed to being inserted in the esophagus. Alternately, it is anticipated that in some embodiments, additional ports are in fluid communication with the manometer/T-piece valve 50 for connection to an external carbon dioxide detector.

Although not shown, it is anticipated that in some embodiments, a bacterial and/or viral filter is inserted in the gas supply path, thereby reducing flow of such agents back into the gas supply path or into the ambient air. When a filter is included, the filter is made from a non-ferromagnetic material.

Although not shown, it is anticipated that in some embodiments, a nebulizer is fluidly inserted in the flow of gas for introducing a liquid mist into the gas. Such nebulizers are known in the industry and often include a nozzle and/or venturi to convert a liquid medication into a mist that is included in the gas supplied to the patient 100. When a nebulizer is included, the nebulizer is made from a non-ferromagnetic material.

Although not shown, it is further anticipated that in some embodiments, an injection port is included in fluid communication with the gas supply to allow injection of a fluid or gas directly to the patient 100 through the patient port 64. When an injection port is included, the injection port is made from a non-ferromagnetic material.

Referring to FIG. 3, a plan view of the disposable resuscitation system 50 in use in conjunction with an infant face mask 8 is shown. In this example, an infant or neonatal face mask 8 is interfaced to the patient port 64. The administrator 110 (e.g. doctor) using the present invention need not look away from the patient 100 to determine gas pressure since the manometer 52 and finger valve 60/62 are at the location of the patient. When no longer needed, the resuscitation system 50 including the finger valve 60/62, the manometer 52, the gas tubing 80 and the pressure relief device 82, as well as the face mask 8, are disposed of according to hospital procedure.

Referring to FIG. 4, a plan view of the disposable resuscitation system 50 in use in conjunction with an infant tracheal tube 6 is shown. In this example, an infant or neonatal tracheal tube 6 is interfaced to the patient port 64. The administrator 110 (e.g. doctor) using the present invention need not look away from the patient 100 to determine gas pressure since the manometer 52 and finger valve 60/62 are at the location of the patient. When no longer needed, the resuscitation system 50 including the finger valve 60/62, the manometer 52, the gas tubing 80 and the pressure relief device 82, as well as the tracheal tube 6, are disposed of according to hospital procedure.

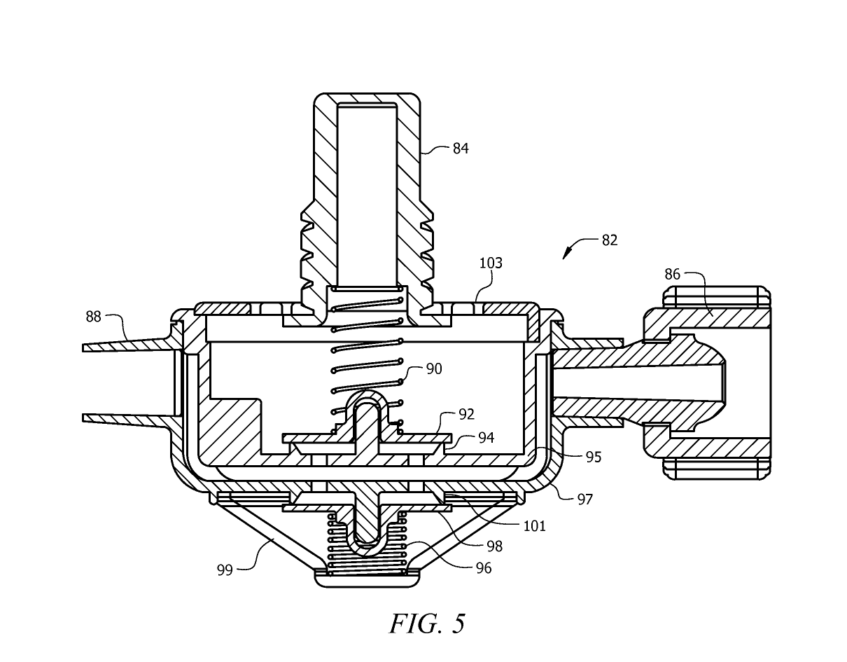

Referring to FIG. 5, a sectional view of a pressure relief device 82 of the disposable resuscitation system 50 is shown. To enable disposability, the pressure relief device 82 is of minimal size, cost, complexity, weight, etc, thereby allowing efficient disposal at minimal cost. The pressure relief device 82 accepts pressurized gas (e.g. air, oxygen) at a, preferably, industry standard gas supply fitting 86. Pressurized gases flow through the pressure relief device 82 and exit to a gas tube fitting 88 that is fluidly coupled to the manometer 52, finger valve 60/62 and patient port 64. It is important to limit the amount of gas pressure injected into a patient's 100 lungs. As pressure backs up from the patient 100 (e.g. the patient's 100 lungs fill), the first pressure relief valve 84/90/92/94 provides an adjustable pressure release. The administrator 110 turns the knob 84 which is threaded in a vented cover 103 of the housing 97 of the pressure relief device 82. As the knob 84 is turned in one direction, by way of a screw action, it screws inwardly into the pressure relief device 82, further compressing the non-ferromagnetic resilient member 90. The more force on the non-ferromagnetic resilient member 90, the more gas pressure needed to overcome the force of the non-ferromagnetic resilient member 90 to vent the gas pressure out between the valve cover 92 and the valve seat 94. As the knob 84 is turned in the opposite direction, the force on the non-ferromagnetic resilient member 90 is abated and less gas pressure is needed to overcome the force of the non-ferromagnetic resilient member 90.

A second valve 96/98/101 is provided as a maximum pressure release should the first valve 84/90/92/94 fail or be adjusted to a dangerous pressure level. The second valve 96/98/101 is housed within a surface 99 that includes vent holes. A second non-ferromagnetic resilient member 96 holds the second valve cover 98 against a second valve seat 101. If the gas pressure exceeds a pre-determined maximum pressure, the gas pressure pushing against the second valve cover 98 overcomes the force of the second non-ferromagnetic resilient member 96, allowing gas to escape out of vent holes in the surface 99 until the gas pressure decreases, at which time the second non-ferromagnetic resilient member 96 has sufficient force as to close the second valve cover 98 against the second valve seat 101. In the example shown, the pressurized air flows between the outer case 97 and an inner case 95 and is routed to the first valve 84/90/92/94 and the second valve 96/98/101.

For proper operation in the vicinity of an MRI system, all components of the pressure relief device 82 are made of a suitable, non-ferromagnetic material such as plastic. This includes the non-ferromagnetic resilient members 90/96, knob 84 and all other components.

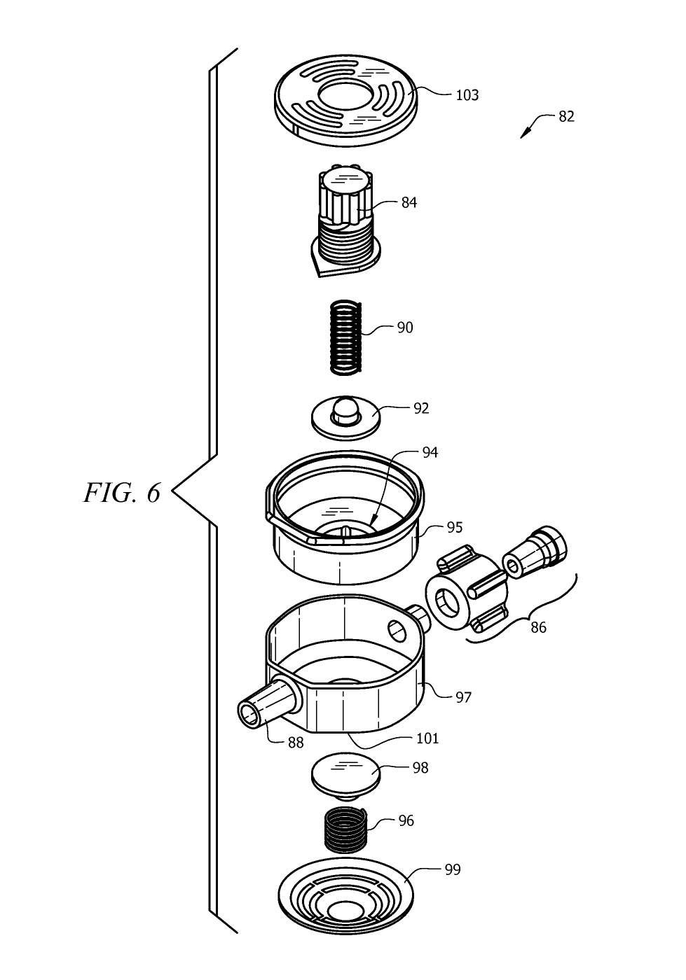

Referring to FIG. 6, an exploded view of a pressure relief device 82 of the disposable resuscitation system 50 is shown. The standard gas supply fitting 86 connects to the outer case 97. The gas tube fitting 88 is connected to or formed on an outer surface of the outer case 97. The first pressure relief valve 84/90/92/94 includes the knob 84 which is threaded in the vented cover 103 of the housing 97. The knob 84 is mechanically interfaced with the non-ferromagnetic resilient member 90, providing adjustable force on the non-ferromagnetic resilient member 90. The non-ferromagnetic resilient member 90 exerts force on the valve cover 92, holding the valve cover 92 against the valve seat 94 until gas pressure forces the valve cover 92 away from the valve seat 94. The second valve 96/98/101 is housed within a surface or cover 99 that also includes vent holes. The second non-ferromagnetic resilient member 96 holds the second valve cover 98 against a second valve seat 101 (not visible).

Again, for proper operation in the vicinity of an MRI system, all components of the pressure relief device 82 are made of a suitable, non-ferromagnetic material such as plastic. This includes the non-ferromagnetic resilient members 90/96, knob 84 and all other components.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A disposable support/resuscitation system for use in the vicinity of an MRI system, the disposable support/resuscitation system comprising:
a pressurized gas inlet for receiving pressurized gas;
a pressure relief device fluidly interfaced to the pressurized gas inlet, the pressure relief device having a first pressure relief valve that opens at a setable gas pressure and the pressure relief device having a second pressure relief valve that opens at a pre-determined maximum gas pressure, the first pressure relieve valve having a first non-ferromagnetic resilient member, the second pressure relief device having a second non-ferromagnetic resilient member;
a manometer fluidly interfaced to the pressure relief valve, the manometer having a third non-ferromagnetic resilient member;
a patient interface port, the patient interface port connected to and in fluid communication with the manometer and with the manually operated valve; and
a finger valve connected to and in fluid communication with the patient interface port, the finger valve having an opening adapted to be selectively blocked by a user's finger such that when a user's finger is placed over the opening, a gas is delivered to the patient interface port for filling the patient's lungs;
whereas the finger valve selectively controls administration of the pressurized gas to the patient interface port and wherein the manometer and the manually operated valve are intended for use in close proximity to the patient.

2. The disposable support/resuscitation system support/resuscitation system of claim 1, wherein the first pressure relief valve is controlled by the rotation of a knob, the knob interfaced to the pressure relief device by threads, wherein turning of the knob in a first direction further increases a force of the first non-ferromagnetic resilient member resulting in a greater force being applied to hold a valve cover against a valve seat, thereby counteracting pressure exerted by gas pressure pushing against an opposing side of the valve cover.

3. The disposable support/resuscitation system of claim 1, further comprising a colorimetric carbon dioxide detector in fluid communication with the patient interface port.

4. A disposable support/resuscitation system for use in the vicinity of an MRI system, the disposable support/resuscitation system comprising:
a pressurized gas inlet for receiving pressurized gas;
a pressure relief device fluidly interfaced to the pressurized gas inlet, the pressure relief device having means for adjustably regulating gas pressure, the pressure relief device having at least one resilient member, the at least one resilient member made from non-ferromagnetic materials;
means for displaying the gas pressure, the means for displaying the gas pressure connected to and fluidly interfaced to the pressure relief device, the means for displaying the gas pressure having a resilient member, the resilient member made from non-ferromagnetic materials;
a patient interface port, the patient interface port connected to and in fluid communication with the means for displaying and with the means for modulating; and
a finger valve connected to and in fluid communication with the patient interface port, the finger valve having an opening adapted to be selectively blocked by a user's finger such that when a user's finger is placed over the opening, a gas is delivered to the patient interface port for filling the patient's lungs;
whereas the finger valve selectively controls administration of the gas pressure to the patient interface port and wherein the means for displaying and the finger valve are intended for use in close proximity to the patient;
whereas disposable support/resuscitation system comprises only non-ferromagnetic materials.

5. The disposable support/resuscitation system support/resuscitation system of claim 4, wherein the means for adjustably regulating the gas pressure includes a control knob, the control knob is interfaced to the pressure relief device by threads, the control knob is interfaced to one of the at least one non-ferromagnetic resilient members, wherein turning of the knob in a first direction increases a force of on the one of the at least one non-ferromagnetic resilient members resulting in a greater force being applied against a valve cover, pushing the valve cover against a valve seat, thereby counteracting pressure exerted by the gas pressure that is pushing against an opposing side of the valve cover.

6. The disposable support/resuscitation system of claim 4, further comprising means for detecting carbon dioxide, the means for detecting carbon dioxide is in fluid communication with the patient interface port.

7. A disposable support/resuscitation system comprising:
a pressure relief device having a gas inlet and a gas outlet, the pressure relief device has a first pressure relief valve that opens at a setable gas pressure;
a manometer in fluid communication with the gas outlet of the pressure relief device;
a patient interface port, the patient interface port molded as an extension of the manometer; and
a finger valve having a body and a cap, the body molded as an extension of the manometer, the cap affixed to the body, the finger valve in fluid communication with the patient interface port, the finger valve having an opening in the cap, the opening adapted to be selectively blocked such that when blocked, a gas is delivered to the patient interface port for filling the patient's lungs;
whereas the finger valve selectively controls administration of pressurized gas from the gas inlet to the patient interface port.

8. The disposable support/resuscitation system support/resuscitation system of claim 7, wherein the first pressure relief valve is controlled by the rotation of a knob, the knob interfaced to the pressure relief device by threads, wherein turning of the knob in a first direction applies a greater force on a non-ferromagnetic resilient member resulting in a greater force being applied to hold a valve cover against a valve seat, thereby counteracting pressure exerted by gas pressure pushing against an opposing side of the valve cover.

9. The disposable support/resuscitation system of claim 7, further comprising a colorimetric carbon dioxide detector, the colorimetric carbon dioxide detector is in fluid communication with the patient interface port.

10. The disposable support/resuscitation system of claim 7, wherein the gas outlet of the pressure relief device is connected to the manometer by a tube.
